(19) United States
(12) Patent Application Publication
(10) Pub. No.: US 2026/0244271 A1
(43) Pub. Date: Aug. 20, 2026

(54) ELECTRONIC DEVICE COMPRISING A FEATURE EXTRACTOR AND CORRESPONDING FEATURE EXTRACTION METHOD

(71) Applicant: Commissariat à l'Energie Atomique et aux Energies Alternatives, Paris (FR)

(72) Inventors: Joe SAAD, Grenoble Cedex 9 (FR); Adrian EVANS, Grenoble Cedex 9 (FR); Ivan MIRO PANADES, Grenoble Cedex 9 (FR)

(73) Assignee: Commissariat à l'Energie Atomique et aux Energies Alternatives, Paris (FR)

(21) Appl. No.: 19/544,704

(22) Filed: Feb. 19, 2026

(30) Foreign Application Priority Data
Feb. 19, 2025 (FR) FR2501705

(51) Int. Cl. G06F 3/01; G06F 18/213

(52) U.S. Cl. CPC G06F 3/015; G06F 18/213

(57) ABSTRACT

An electronic device including a primary sensor configured to carry out the acquisition of a measurement signal of at least one physiological parameter of a living being; a feature extractor; a primary decoder using the extracted feature and configured to determine a control signal for an actuator with which the living being interacts. The feature extractor is configured to carry out the extraction of features from the measurement signal in an approximate manner at several levels of precision, based on a precision control parameter adjusted according to a value of a quality evaluation parameter. A method for extracting features is also disclosed.

### TECHNICAL FIELD

The technical field of the invention concerns direct neural interfaces, commonly referred to as «BCI» (Brain Computer Interface), intended to control, on the basis of neurophysiological signals, an actuator, as well as any other embedded or implantable system implementing a processing chain comprising a feature extraction step.

### STATE OF THE ART

The direct neural interfaces enable users to control actuators by thoughts. Such interfaces may be intended for people with disabilities or other users. To this end, electrophysiological signals emitted by the cortex are detected and recorded, then processed or decoded by algorithms making it possible to generate a control signal for controlling actuators. The control signal may enable the driving of an exoskeleton, a computer, or a robot, in order to provide user assistance, speech decoding, or even stimulation of the user's muscles.

The implemented algorithms make it possible to translate a user-given instructions, which are captured by electrodes in the form of so-called electrophysiological signals, as they are representative of the electrical activity of neurons. This electrical activity may be measured at the cortical level by means of cortical electrodes arranged within the cranial cavity. It may also be measured by electroencephalography electrodes, which are less intrusive since they are placed on the scalp, but also less efficient, particularly in terms of spatial resolution. Another solution consists in recording electrophysiological signals by magnetoencephalography, which requires a dedicated installation.

The algorithms implemented are generally based on a predictive model.

The predictive model uses input data, obtained by preprocessing the recorded electrophysiological signals in order to perform feature extraction, so as to generate a control signal for the actuator or actuators.

The observation data or extracted features are generally multidimensional, and may comprise, for example:
a spatial component, representative of the spatial origin of the electrophysiological signal;
a frequency component, representative of the intensity of the electrophysiological signal in different frequency bands;
a temporal component corresponding to a temporal sample of the electrophysiological signal within which the frequency analysis is performed.

The observation data or extracted features may also reflect other information, such as, for example, phase shifts between signals, correlations, intrinsic properties of a signal, such as variance or chaotic parameters.

Each item of observation is associated with an epoch, that is to say, a temporal sample of predetermined duration, after the user has had the intention to perform the task. For each epoch, an observation tensor gathering the observation data is formed. The observation tensor is used to feed a predictive model. The predictive model, when applied to the observation tensor, allows to estimate a control signal, enabling the command of the actuators. The control signal is generally expressed as a control vector.

One of the main challenges in the development of BCIs, as well as in many other wearable or implantable systems using a feature extraction, lies in optimizing energy consumption, particularly for wearable and implantable applications. Indeed, the processing chain, comprising feature extraction, decoding, and generation of a control signal, is highly energy-consuming, the feature extraction being a particularly energy-consuming part of this chain.

Solutions have been proposed to limit energy consumption, for example in the document by U. Shin et al., “NeuralTree: A 256-Channel 0.227-uJ/Class Versatile Neural Activity Classification and Closed-Loop Neuromodulation SoC”, November 2022, doi: 10.1109/JSSC.2022.3204508. This document discloses a system-on-a-chip (SoC) for neural activity classification and closed-loop neuromodulation. In this system, the selection of extracted features is made once at the time of model learning, during the training phase. A decision tree is built, and at each node of the tree, a subset of features is used. The selection of the extracted features at each node is made so as to minimize energy. This system therefore implements a predetermined static approach defined during training, which binary-defines the use or non-use of a feature and does not allow dynamic adaptation to the operating conditions of the system.

The present invention aims to remedy all or part of the drawbacks mentioned above.

### SUMMARY OF THE INVENTION

To this end, the present invention relates to an electronic device comprising:
a primary sensor configured to acquire a measurement signal of at least one physiological parameter of a living being;
a feature extractor configured to extract a feature from the measurement signal;
a primary decoder using the extracted feature and configured to determine a control signal;
an actuator with which the living being interacts;
an actuator drive module configured to drive the actuator as a function of the control signal determined by the primary decoder.

The feature extractor is configured to be able to perform feature extraction from the signal measurement in an approximate manner according to a plurality of precision levels, the required precision level being a function of a desired precision control parameter, the electrical power consumption of the feature extractor increasing with the precision level;

The device further comprises a control module configured to determine a quality evaluation parameter of the action of the actuator in interaction with said living being;

The control module is configured to adjust the precision control parameter as a function of a value of the quality evaluation parameter.

According to one possibility, said selected precision control parameter corresponds to the lowest precision level that allows to cause the value of the quality evaluation parameter to tend towards a predefined quality value range or to maintain the value of the quality evaluation parameter within a predefined quality value range.

Thanks to the arrangements according to the invention, a dynamic control of the quality of the features extracted from the measurement signals is achieved by means of a feedback loop, allowing continuous adjustment so as to reduce the computational complexity when high-quality features are not required and thereby reduce the overall power consumption of the device. This approach is applicable when the computational error incurred has little influence on the system output. This approach aims to maximize energy efficiency while maintaining acceptable operation of the overall system, thereby enabling more portable and less energy-consuming applications.

This approach allows the quality of the extracted features to be adapted according to the task requirements. For example, for simple tasks requiring lower precision, the device according to the invention may reduce the quality of the extracted features, thereby achieving significant energy savings. For more complex tasks requiring high precision, the device may increase the quality of the extracted features, thus ensuring a sufficient performance.

The modification of the quality may be performed during operation of the circuit. It is not necessary to stop the circuit to change it. The operation of the other elements in the processing chain is not affected.

According to various possibilities, the precision levels may be continuous or discrete precision levels.

The term «precision level» means the precision level of the computation methods or, in other words, the level of approximation of the methods relative to a full-precision or exact computation.

The expression «causing the value of the quality evaluation parameter to tend toward a predefined quality value range or maintaining the value of the quality evaluation parameter within a predefined quality value range» means that the adjustment of the precision control parameter aims to include the quality evaluation parameter within the quality value range. Thanks to these arrangements, a regulation of the precision control parameter as a function of the quality evaluation parameter is achieved. The selection of the value of the precision control parameter may be carried out iteratively. The objective of maintaining the value within the quality value range may result in the quality evaluation parameter temporarily leaving this range during regulation, in particular in order to minimize the value of the precision control parameter.

According to one possibility, the precision control parameter is computed as a continuous or discrete function of the quality evaluation parameter.

According to one possibility, the feature extractor may perform feature the extraction from the measurement signal either in full precision or according to a precision level, full-precision extraction corresponding to an exact computation.

According to one possibility, the quality value range is defined by a determined deviation between the current quality evaluation parameter and the quality evaluation parameter that would be obtained with a full-precision computation.

According to one possibility, the measurement signal is an electrophysiological signal, in particular an electrophysiological signal emitted by the cortex.

According to one possibility, the device comprises an integrated circuit comprising at least one of the following: the feature extractor, the control module, and the primary decoder.

According to one possibility, the actuator is an element selected from a spinal cord stimulator, a muscle stimulator, an exoskeleton actuator, a speech generator, a wheelchair actuator, such as a wheelchair motor, a display.

According to one possibility, the feature extractor is configured to perform an approximate computation of a convolution between the input signal and a reference signal, taking into account the precision control parameter.

According to various possibilities, the reference signal may be a wavelet, a trigonometric function, filter coefficients, or a signal allowing the computation of Fourier coefficients.

This technique allows for the extraction of a spectro-temporal information.

According to one possibility, the approximate computation uses an approximation technique of the continuous wavelet transform type, piecewise linear.

According to another possibility, the approximate computation uses an approximation technique of the quantized wavelet transform type.

According to another possibility, the approximate computation uses a discrete cosine transform.

According to another possibility, the approximate computation uses a continuous wavelet transform with an adaptive quantization level.

According to another possibility, the approximate computation uses a fast Fourier transform with adaptive resolution.

According to another possibility, the approximate computation uses digital filters with variable-size supports.

According to another possibility, the approximate computation uses an adjustment of the numerical precision, in particular the number of bits,

According to another possibility, an approximate arithmetic or other approximate computing approaches may be used by adapting the approximation level to a value of the precision control parameter.

According to another possibility, the precision control parameter corresponds to a precision level of an approximation function of the reference signal.

According to one possibility, the precision level corresponds to the number of points used to approximate a wavelet.

According to one possibility, the feature extractor is configured to extract a set of features from the measurement signal, the control module being configured to determine a single precision control parameter for a plurality of features extracted from the set of features, or to determine an individual precision control parameter for each of the features extracted from the set of features.

According to one possibility, the control module is configured to determine a single quality evaluation parameter for a plurality of features extracted from the set of features, or to determine an individual quality evaluation parameter for each of the features extracted from the set of features.

According to one possibility, the control module may be configured to adjust the precision control parameter for a plurality of features as a function of the same quality evaluation parameter.

According to one possibility, the set of extracted features corresponds to a plurality of frequencies and/or a plurality of spatial locations.

The plurality of extracted features may correspond to a plurality of frequencies and/or a plurality of spatial locations, corresponding, for example, to the positioning of a plurality of primary sensors or sub-parts of the primary sensor. Several extracted features may also correspond to specific time instants.

According to one embodiment, the electronic device comprises a secondary decoder configured to provide a user satisfaction signal, the quality evaluation parameter taking into account the user satisfaction signal.

According to one embodiment, the secondary decoder for the user satisfaction level is configured to decode features extracted from electrophysiological signals emitted by the cortex.

According to another possibility, the secondary decoder is configured to perform a decoding based on the input signals x, or simple features that are easy to extract from these signals, such as the mean and/or the variance of these signals.

The decoding may be performed in real time.

According to one embodiment, the quality evaluation parameter is the user satisfaction signal.

According to one possibility, the user's satisfaction level is decoded while the user is performing a task. If, during the decoding, the decoder identifies that the user is not satisfied, the control module is configured to progressively increment the precision control parameter, and thus the precision of the computations, in order to reduce the decoding error.

Thus, this signal may be used to dynamically control the quality of the extracted features.

According to one embodiment, the control module is configured to periodically perform or control an exact feature extraction computation and to measure a deviation between the result obtained with an exact feature extraction and that obtained with an approximate feature extraction using the level of the current extraction precision control parameter, so as to determine a value of the quality evaluation parameter.

According to one possibility, the deviation may be evaluated by a correlation of the exact and approximate extracted features.

According to another possibility, the deviation may be evaluated by comparing the decoding result obtained, for example by identifying whether the decoding result is the same with the approximate extracted features and the exact extracted features, in particular with respect to the determination of a control signal or an output class.

According to one embodiment, the control module is configured to determine the quality evaluation parameter by taking into account a value of a performance indicator, the device comprising a secondary measurement sensor configured to acquire a control quantity or a user interface configured to collect a control information from a user, the control module being configured to determine the quality evaluation parameter by taking into account the performance indicator determined from a value of the control quantity or from the control information.

According to these arrangements, the performance indicator corresponds to an objective «ground truth» associated with a sensor measurement or with user feedback.

According to various examples, a performance indicator may be a decoding success rate, a precision of the performed actions, or the variability of the values obtained during the decoding.

According to one possibility, if the performance decreases, the control module may increase the quality of the extracted features in order to improve the precision.

According to one embodiment, the secondary measurement sensor is a position sensor, or a plurality of position sensors configured to identify abnormal positions of elements controlled by the actuator.

According to one embodiment, the secondary measurement sensor is a motion sensor, or a plurality of motion sensors configured to analyze the movement of actuator-controlled effectors, such as the exoskeleton, or the user's limbs, and to discriminate deviations from known scenarios such as walking, running, or grasping an object.

According to one embodiment, the user interface is a user interface sensor, such as, for example, a rheostat similar to a volume knob, allowing the user to directly adjust the quality according to their perception.

According to one embodiment, the control module is configured to determine the quality evaluation parameter by taking into account an anomaly detection based on the value of the control signal and the value of the extracted features.

According to one embodiment, the error analysis is performed by frequency band or by extracted feature. Thus, the control module is configured to identify the frequencies or features involved in a given task that require improvement. According to one possibility, the control module may increase the quality of the extracted features for specific identified frequencies by adjusting the precision control parameter.

According to one embodiment, the anomaly detection may be based on a comparison between the values or amplitudes of the extracted features and a reference of expected values.

According to one embodiment, the electronic device comprises a reference database comprising references of expected value for the extracted feature values in relation to values of the control signal.

According to one embodiment, the control module is configured to determine the quality evaluation parameter by taking into account a value of a confidence level or probability associated with a result of the primary decoder.

These arrangements make it possible to use the probabilities provided by the primary decoder as a confidence level in the issued decision. For example, decoders such as linear classifiers or decision trees associate a probability with the selected majority class. This probability may be used to control the quality level of the extracted features.

According to one possibility, the control module may be configured to associate the confidence level or probability of a decoded class with a quality level required for the subsequent decoding, and thus to determine at each step the precision control parameter of the computed extracted features.

According to another possibility, the control module may be configured to determine a quality of the decoder decision and incrementally adapt the precision is control parameter of the computed extracted features to the determined quality.

The quality of the decision may be estimated, for example, by thresholding the decision probability. If the decision exhibits a high confidence level—and may therefore be considered over-satisfactory—the control module may be configured to incrementally decrease the feature extraction precision control parameter. Conversely, if the decision exhibits a low level of confidence—and may therefore be considered doubtful—the control module may be configured to incrementally increase the feature extraction precision control parameter.

According to one possibility, the control module is configured to determine the extraction precision control parameter by taking into account:
a set of possible ordered configurations of the extraction precision control parameter;
a current value of the extraction precision control parameter selected from the set of possible configurations of the precision control parameter;
an increment dependent on the quality control parameter defining the transition from the current value of the precision control parameter to a new value of the precision control parameter selected from the ordered set of possible configurations of the precision control parameter.

According to one embodiment, the control module is configured to determine the extraction precision control parameter by taking into account:
a current value of the extraction precision control parameter;
a modification of the extraction precision control parameter, for a subset of randomly or predefined extracted features or features based on learned or predefined heuristics depending on the quality control parameter.

The present invention also relates to a method for extracting features from a measurement signal of at least one physiological parameter of a living being, comprising:
A step of acquiring the measurement signal of at least one physiological parameter of a living being;
A step of extracting features from the measurement signal;
A decoding step using the extracted features in order to determine a control signal of an actuator with which the living being interacts;
A step of driving the actuator as a function of the control signal

wherein the step of extracting features from the measurement signal is performed in an approximate manner by taking into account a precision control parameter, the method further comprising:
A step of determining a quality evaluation parameter;
A step of adjusting the precision control parameter as a function of a value of the quality evaluation parameter by selecting said precision control parameter corresponding to the lowest precision level that makes it possible to cause the value of the quality evaluation parameter to tend toward a predefined quality value range or to maintain the value of the quality evaluation parameter within a predefined quality value range.

### BRIEF DESCRIPTION OF THE FIGURES

The invention will be better understood from the following detailed description, given with reference to the accompanying drawing in which:

FIG. 1 represents a first embodiment of an electronic device according to the invention;

FIG. 2 represents a second or a third embodiment of an electronic device according to the invention;

FIG. 3 represents a fourth embodiment of an electronic device according to the invention;

FIG. 4 represents a fifth embodiment of an electronic device according to the invention;

FIG. 5 represents a sixth embodiment of an electronic device according to the invention;

FIG. 6 illustrates the technique of approximating a wavelet by continuous piecewise linear approximation and its use for approximating a convolution of a signal by a wavelet;

FIG. 7 shows a comparison of two wavelet approximations.

FIG. 8 represents the correlation of approximately extracted features relative to the exact features.

FIG. 9 represents the evolution of the precision obtained as a function of the energy consumed for different values of the precision control parameter for a 5-class decoding relating to the selection of a finger of the hand.

FIG. 10 represents the evolution of the precision obtained as a function of the energy consumed for different values of the precision control parameter for a 2-class decoding relating to the determination of a rest state or activity state.

FIG. 11 shows the influence of the signal quality on the correlation between a continuous wavelet transform (CWT) and a piecewise linear continuous wavelet transform (PLCWT).

FIG. 12 illustrates the actual decoded value for 5 features and their expected value ranges, in a given state.

FIG. 13 represents a flowchart of a method for extracting features according to the invention.

### DETAILED DESCRIPTION OF THE INVENTION

In the detailed description that follows with reference to the figures defined above, identical elements or elements fulfilling identical functions may retain the same reference signs in order to simplify understanding of the invention.

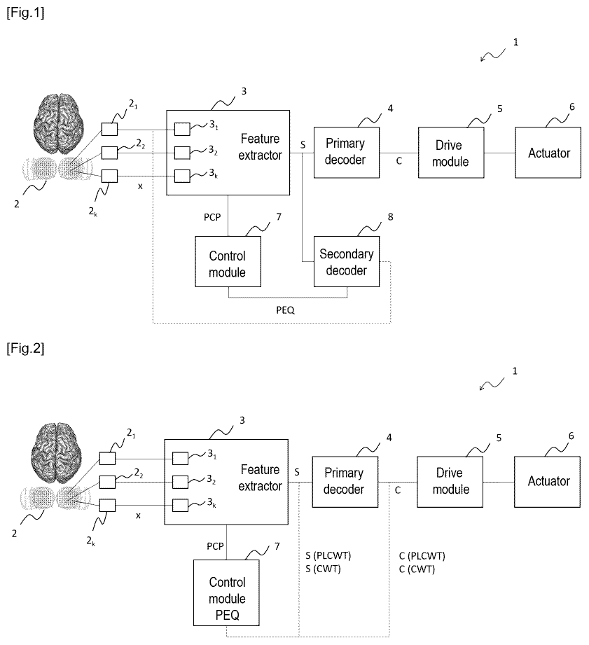

FIGS. 1 to 5 show different embodiments of an electronic device 1 comprising a set of primary sensors 2 configured to acquire a first set of measurement signals x of at least one physiological parameter of a living being. In particular, according to the embodiments shown in the figures, the set of primary sensors 2 may be a set of electroencephalography (EEG) electrodes, electrocorticography (ECoG) electrodes, or a microelectrode array (MEA). In the following, an example of a living being will be considered that is a human user, referred to as the user.

The first set of measurement signals x is a set of electrophysiological signals, in particular electrophysiological signals emitted by the cortex, or neuronal signals or brain network signals.

The different types of sensors exhibit different spatial resolutions depending on the degree of invasiveness. Although both ECoG and EEG electrodes capture signals that are averaged over a population of neurons, the ECoG signals have better spatial resolution. Both MEA and ECoG arrays are implanted surgically; however, the procedure is less invasive in the latter case, since the array is placed on the surface of the brain.

Previous studies have shown that ECoG signals exhibit frequency modulations that are coupled to specific mental tasks such as motor intention.

Furthermore, the brain signals are non-stationary, meaning that useful information can also be extracted in the time domain.

The electronic device 1 comprises a feature extractor 3 configured to extract a second set of features S from the first set of measurement signals x. The feature extractor (3) is configured to perform feature (S) extraction from the measurement signal (x) either approximately or in full precision according to several precision levels, the required precision level being a function of a desired precision control parameter (PCP), the electrical power consumption of the feature extractor increasing with the precision level.

According to one embodiment, a wavelet transform (WT) may be used to perform this extraction, since it makes it possible to extract features containing information in the time-frequency domain, or in other words, spectro-temporal information.

The computation of a wavelet transform, more generally known as a continuous wavelet transform (CWT), requires a convolution between the analyzed signal x and a wavelet ψₐ or its complex conjugate

$${ψ}_{a}^{*} .$$

This approach is computationally expensive and leads to high energy consumption, since decoding requires the extraction of these features on multiple electrodes, for example between 16 and 256, and across multiple frequency bands, for example between 1 and 24. It is therefore necessary to perform a convolution or correlation per electrode and per frequency band. These features are then used by the decoder for decision-making.

The continuous wavelet transform CWT can be expressed as follows:

$$S ( τ ) = {\int }_{K} x ( t + τ ) {ψ}_{a}^{*} ( t ) dt \qquad ( E1 )$$

where a is the scale factor for a given frequency, τ is the temporal shift of the convolution, and K is the interval over which the wavelet is defined. For digital signals, the interval is discretized, and the integral becomes a finite sum. The discretized transform WT is therefore expressed as an inner product.

$$S ( τ ) = {\sum }_{i = 1}^{N} x ( i + τ ) {ψ}_{a}^{*} ( i ) \qquad ( E2 )$$

Thus, the extraction circuit 3 is configured to perform feature extraction from the measurement signal using an approximate computation taking into account the precision control parameter PCP in order to reduce the number of computations to be performed.

In particular, when the extraction mode corresponds to the computation of wavelet transforms, that is, a convolution between the input signal and a wavelet, taking into account a precision control parameter, it is possible to use an approximate computation mode based on a piecewise linear continuous wavelet transform (PLCWT).

The piecewise linear continuous wavelet transform (PLCWT) technique is based on a first-order piecewise approximation φ of the wavelet

$${ψ}_{a}^{*} ,$$

as shown in FIG. 6. The wavelet interval is divided into J−1 segments, and on each subdivision segment i, the wavelet is approximated by a linear function φᵢ. The set of pieces of linear functions thus connects J points.

Applying integration by parts to the definition of CWT in its integral form, using this approximation, yields the following expression (E4):

$$S ( τ ) = {X}^{( 1 )} ( {a}_{J} + τ ) {ϕ}_{J - 1} ( {a}_{J} ) - {X}^{( 1 )} ( {a}_{1} + τ ) {ϕ}_{1} ( {a}_{1} ) - {\int }_{{a}_{1}}^{{a}_{J}} {X}^{( 1 )} ( t + τ ) {ϕ}^{′} ( t ) dt \qquad ( E4 )$$

Where X⁽¹⁾ denotes the first antiderivative of the signal and φ′ the derivative of the piecewise approximated wavelet φ. The last integral term, denoted I(τ), is then computed on the subdivision segments on which the wavelet approximation is piecewise linear. The derivative φ′₁ on each segment is constant and may be removed from the integral as shown in E4′).

$$I ( τ ) = {\sum }_{i = 1}^{J - 1} {ϕ}_{i}^{′} {\int }_{{a}_{i}}^{{a}_{i + 1}} {X}^{( 1 )} ( t + τ ) dt \qquad ( {E4}^{′} )$$

$$I ( τ ) = {\sum }_{i = 1}^{J - 1} {ϕ}_{i}^{′} [ {X}^{( 2 )} ( {a}_{i + 1} + τ ) - {X}^{( 2 )} ( {a}_{i} + τ ) ]$$

$$I ( τ ) = {\sum }_{i = 1}^{J} {b}_{i} {X}^{( 2 )} ( {a}_{i} + τ )$$

X⁽²⁾ is the second antiderivative of the signal and the reordered coefficients bᵢ of the sum are given as follows:

$${b}_{i} = \{ \begin{matrix}{b}_{1} = - {ϕ}_{1}^{′} \\ {ϕ}_{i - 1}^{′} - {ϕ}_{i}^{′} pour i ∈ \text{?} 2 , J - 1 \text{?} \\ {b}_{J} = {ϕ}_{J - 1}^{′}\end{matrix}$$

In practice, to compute this convolution, the signal is integrated twice using trapezoidal or rectangular integration, which has a negligible computational cost compared with the other processing steps. Letting L be the signal length, and recalling that J is the number of points used to approximate the wavelet, the computation requires L×J additions and multiplications to evaluate the sum in (E4′). Additional optimizations are possible depending on the selected type of wavelet.

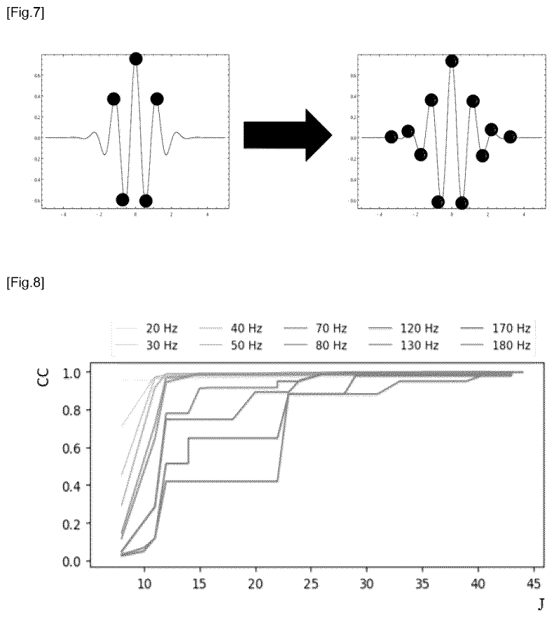

FIG. 7 shows two possible approximations of a wavelet using this technique. In the left diagram of FIG. 7, the function is discretized using very few points. The significant variations of the wavelet are centered in a neighborhood around zero. The approximation points are therefore mainly selected in this area. The distribution may be widened by adding points toward the extremities, as shown in the right diagram of FIG. 7. This discretization is used to compute the convolution with the input signal x. By decreasing the number of subdivision points, the number of required computations is reduced. If the wavelet approximation on the left of FIG. 7, with few points, is used, the correlation with the theoretical result (CWT) using the non-approximated wavelet will be lower than the right version using more points.

The choice of the number of points used to approximate a wavelet is an example of a precision control parameter PCP that makes it possible to influence the quality of the extracted features S.

The feature extractor 3 is thus configured to perform feature S extraction from the measurement signal x while taking into account a precision control parameter PCP. In the case of the PCWT technique, the precision control parameter corresponds to the number of subdivision points set to approximate the wavelet in a piecewise manner.

For the BCI decoding, it is necessary to extract information at different frequencies. According to one possibility, the extractor may comprise a single feature extraction module that extracts features sequentially, one by one. This may be the case if a microcontroller is used for extraction. In another approach, the feature extraction circuit 3 comprises a plurality of extraction modules 3k configured to extract features S. It may also comprise distinct modules for processing input signals x originating from different electrodes 2k. According to different embodiments according to the second possibility, the number of feature extractors may range from N, when N different types of features are extracted, up to at most N×K, where K is the number of electrodes. For the different extracted frequencies, the same computation module may be used by changing the module's multiplicative coefficients.

The plurality of extracted features S corresponds to a plurality of frequencies and/or a plurality of spatial locations, corresponding for example to the positioning of the electrodes. Several extracted features S may also correspond to specific time instants τ.

Thus, wavelets of different frequencies are used. A high-frequency wavelet will have a greater number of peaks per time interval than a low-frequency wavelet. To approximate high-frequency wavelets, more points are therefore required than for low-frequency wavelets in order to obtain approximations of comparable quality.

One way to evaluate the quality of the approximated extracted features (S) is by the level of correlation with the exact features. In particular, this correlation may be obtained by periodically performing an exact computation, for example, every M iterations (for example 1000), allowing for regular verification. The correlation measurement may be performed separately for each frequency band and for each electrode. By acting on the correlation level for each frequency, this enables fine and precise adaptation of the quality of the extracted features, depending on the task requirements and on signal variability between different patients or for a same patient over time. For example, for frequencies that contain little information but are nevertheless useful or necessary for the task, the correlation level may be reduced, thereby enabling additional energy savings while retaining the useful extracted information. For frequencies more important for the task, the correlation level may be increased, thereby ensuring optimal performance.

FIG. 8 shows the correlation between the computed and exact features. Each row corresponds to a different wavelet frequency. The abscissas represent the number of subdivision points J used to approximate the wavelet using the PLCWT technique. The ordinates represent the correlation coefficient CC. On the right-hand side of the figure, it can be observed that using 45 subdivision points makes it possible to obtain a correlation coefficient of 1, that is to say the same value, up to an affine transformation, as that obtained using exact CWT wavelets. On the left side of the figure, it can be observed that with only 10 approximation points, the correlation coefficient values are lower, thus indicating a poorer approximation quality. In the middle of the figure, with 20 subdivision points, it can be observed that low frequencies (<70 Hz) provide higher correlation levels than high frequencies (>120 Hz).

Various other techniques may be used instead of the PLCWT technique described above to extract features S in an approximated manner while adapting the result quality using a precision control parameter PCP.

Thus, according to a first possibility, a discrete cosine transform may be used. In this case, the precision control parameter PCP may correspond to the number of points used by the discrete cosine transform or to the numerical precision, that is to say, the number of bits used for encoding the information.

According to another possibility, a continuous wavelet transform with adaptive quantization, of the QCWT type (also referred to as quantized CWT), may be used. In this case, the precision control parameter PCP may correspond to the numerical precision of the wavelet approximation.

According to another possibility, a fast Fourier transform with adaptive resolution may be used, the resolution being usable as the precision control parameter PCP. Indeed, reducing the number of points in a fast Fourier transform reduces the frequency resolution but also the number of arithmetic operations and therefore the computation energy consumption.

According to another possibility, digital filters with variable-size supports can be used. The quality of a digital filter, for example, a filter of the finite or infinite impulse response type, depends on the number of coefficients of the filter. Thus, the extractor can have several precomputed filters with different numbers of coefficients. The precision control parameter PCP allows the selection of a filter from among the plurality of filters as a function of the desired quality.

According to another possibility, an adjustment of the numerical precision, in particular of the number of bits, can be used in the computations. When the number of bits used to represent a signal is reduced, a quantization noise is introduced. The number of bits of precision can be modified on the fly, as a function of a precision control parameter PCP, as needed.

According to another possibility, an approximate arithmetic or other approximate computation approaches can be used by adapting the level of approximation to a value of the precision control parameter PCP. Approximate computing methods are described, for example, in the paper by M. Shafique et al., DAC '16: Proceedings of the 53rd Annual Design Automation Conference, Article No.: 99, Pages 1-6 (e.g. https://dl.acm.org/doi/10.1145/2897937.2906199).

The electronic device 1 comprises a primary decoder 3 or decoding circuit.

The primary decoder 3 is configured to use the extracted features S produced by the feature extractor 3 to determine a control signal C. The primary decoder 3 can notably use a decoding model such as the support vector machines, decision tree forests or artificial neural networks.

Most decoding models that can be used by the decoding circuit are insensitive to affine transformations of the inputs. Therefore, a high level of linear correlation between the approximate and theoretical features is sufficient to ensure that the decoder have good performance. However, there may be cases where the decoding precision is high despite the fact that the used features are weakly correlated with the theoretical features.

Indeed, the correlation level of an approximation is an indicator of the quality of the obtained information. However, a more reliable metric for evaluating the system is the decoding precision.

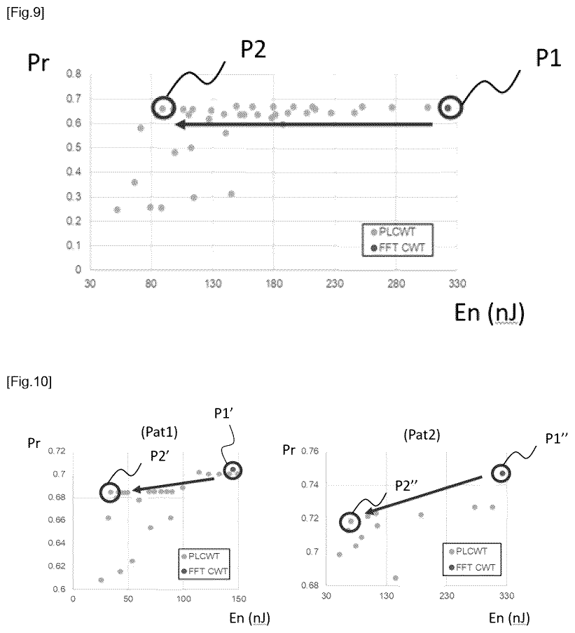

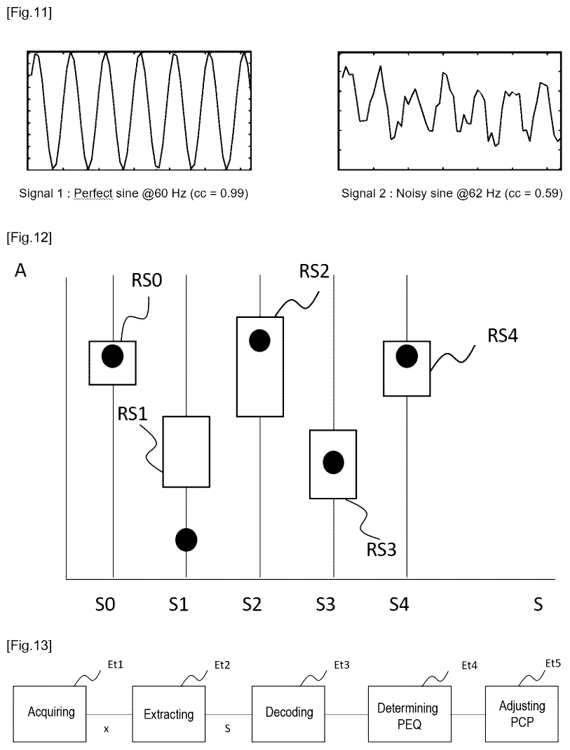

For example, for a 5-class decoding task corresponding to a user finger selection, the obtained precision Pr with several choices of quality parameters, and in particular of subdivisions J to approximate the wavelets, can be computed, in relation to the energy required Eₙ in nJ to evaluate the convolution for each selected subdivision. FIG. 9 shows the results of these computations according to an embodiment. We observe that there are approximations that make it possible to maintain the same precision (within 1%), while consuming 3.5 times less energy by comparing the point P1 of the graph, which corresponds to a CWT-type transform carried out by a fast Fourier transform, and the point P2, which uses a PLCWT approximation.

According to another example illustrated in FIG. 10, for a 2-class decoding task corresponding to a determination of a rest or active state, the consumed energy can be reduced by a factor of 4 with a 2% loss in precision for a first user Pat1 by comparing the point P1′ on the graph, which corresponds to a CWT-type transform carried out by the Fast Fourier Transform, and the point P2′, which uses a PLCWT approximation and by a factor of 5 with only 3% loss in precision for a second user Pat2 by comparing the point P1″ on the graph, which corresponds to a CWT-type transform carried out by the FFT, and the point P2″ which uses a PLCWT approximation. It is important to note that there are many intermediate approximations, that allow an acceptable trade-off between the energy gain and the precision loss.

In summary, we can find wavelet approximation values that make it possible to reduce computational complexity while maintaining a high decoding precision. We reiterate that the PLCWT technique is just one example of a feature whose quality can be modulated.

The electronic device 1 also comprises an actuator 5 with which the living being interacts, which may be, in particular, a spinal cord stimulator, a stimulator of the user muscles such as, for example, a stimulator used in functional electrical stimulation FES treatments, an exoskeleton actuator, a speech generator, a wheelchair actuator, such as a wheelchair motor, a display configured to display information decoded from the user neural signals. The device further comprises an actuator driving circuit configured to drive the actuator as a function of the control signal C of the primary decoder 4.

The device further comprises a control module 7 configured to measure or collect a quality evaluation parameter PEQ of the action of the actuator in interaction with said living being, the control module being further configured to adjust the precision control parameter PCP as a function of a value of the quality evaluation parameter PEQ.

The control module 7 is configured to adjust the precision control parameter PPPCP as a function of a value of the quality evaluation parameter (PEQ) by selecting said precision control parameter PCP corresponding to the lowest level of precision allowing the value of the quality evaluation parameter to tend towards a predefined range of quality values or to maintain the value of the quality evaluation parameter within a predefined range of quality values.

The quality evaluation parameter PEQ and the range of quality values can be determined in several ways according to several embodiments described below.

According to a first embodiment as shown in FIG. 1, the electronic device 1 comprises a secondary decoder 8 for the user satisfaction level configured to provide a user satisfaction signal, the quality evaluation parameter PEQ taking into account this user satisfaction signal (or being, for example, equal to this satisfaction signal). In this case, the predefined range of quality values may correspond to values above a minimum user satisfaction value (or to a simple positive satisfaction value if the satisfaction signal is binary). By way of example, the used secondary decoder may be of the type described in the patent application filed under No. FR2415312. According to one possibility, the secondary decoder 8 for the user satisfaction level is configured to carry out decoding of extracted features S of electrophysiological signals emitted by the cortex (or neuronal signals) provided by the feature extractor 3. The decoding may be carried out in real time.

According to one possibility, the secondary decoder is configured to carry out decoding based on the input signals x, or on simple features to be extracted from these signals, such as the mean and/or the variance of these signals, which may be sufficient to decode a satisfaction level. Thus, the input signals x can be processed directly by the secondary decoder or by a simple auxiliary module.

According to another possibility, it would be possible to configure the secondary decoder to take into account both the features S extracted by the feature extractor 3 and the simple features, for example of mean or variance type, obtained directly from the input signal x.

According to one possibility, the user satisfaction state is decoded while the user executes a task. If, during decoding, the secondary decoder identifies that the user is not satisfied, the control module 7 is configured to modify the precision control parameter PCP in order to progressively increase the precision of the computations and improve the value of the quality evaluation parameter PEQ, that is to say the user satisfaction. Thus, this signal can then be used to dynamically control the quality of the features.

The secondary decoder 8 may be configured to operate at a lower frequency than the main decoder and/or by performing less computation than the main decoder.

The criterion used to determine the computation power and frequency usable by the secondary decoder 8 can be defined by comparing:
the power PP consumed by the main processing chain configured to determine the command C comprising the sensors, the feature extractor 3, the primary decoder 4 with the current precision control parameter PCP;
the power PPF consumed by the processing performed by the main processing chain configured to determine the command C comprising the sensors, the feature extractor 3, the primary decoder 4 at full precision;
the energy ES consumed by the feedback chain comprising the secondary decoder, the control module 7 and/or the other elements aimed at determining the quality evaluation parameter PEQ and modifying the precision control parameter PCP.
the frequency FS at which the feedback chain processing is performed.

Thus, the criterion can be defined as follows:

$$ES \times FS + PP < PPF \qquad ( E5 )$$

This energy criterion or relationship E5 reflects the fact that the energy or the power consumed by the main chain and the secondary chain, using an approximation corresponding to a current value of the precision control parameter PCP, is lower, and preferably much lower, than the energy or the power consumed by the main chain at full precision. This relationship is applicable to the first embodiment presented above, but also to all the embodiments presented below.

According to a second embodiment shown in FIG. 2, the control module 7 is configured to periodically carry out an exact feature S extraction computation or to control a feature extractor 3 in order to periodically carry out an exact feature S extraction computation. Following the exact computation, the control module is configured to measure the deviation between the result obtained with an exact feature extraction and that obtained with approximate feature S extraction, using the level of the current extraction precision control parameter PCP, in order to determine a value of the quality evaluation parameter PEQ. According to one possibility, the deviation can be evaluated by computing a correlation CC of the exact and approximate extracted features.

According to another possibility, the deviation can be evaluated by comparing the result of the decoding carried out by the primary decoder, for example by identifying whether the result of the decoding is the same with the approximate extracted features and exact extracted features, particularly with respect to the determination of an output class.

The exact computation is carried out, for example, for one epoch out of 1000.

In this embodiment, the predefined range of quality values can correspond to a maximum value of the permissible deviation.

The precision control parameter PCP is computed as a continuous or discrete function of the quality evaluation parameter PEQ. FIG. 8 shows the influence of the signal quality on the correlation coefficient (CC) between CWT and PLCWT, for a transform using a (theoretical and approximate) 60 Hz wavelet.

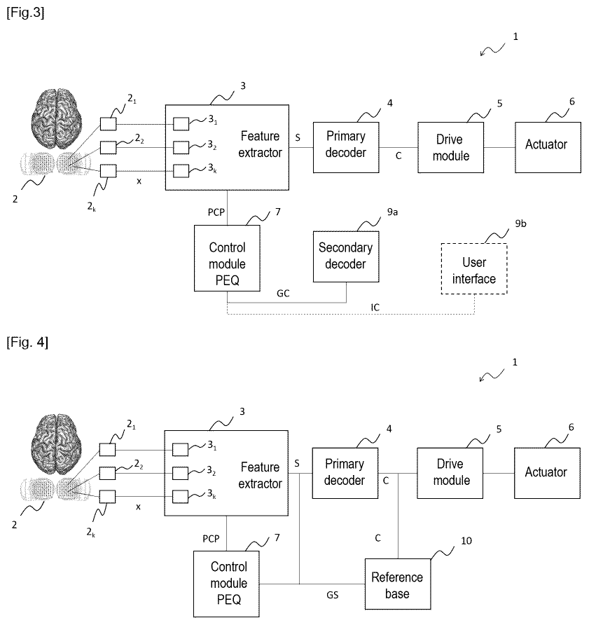

According to a third embodiment shown in FIG. 3, the control module 7 is configured to determine the quality evaluation parameter PEQ by taking into account a value of a performance indicator corresponding to a “ground truth”. According to various examples, a performance indicator could be a decoding success rate, a precision of the performed actions, or the variability of the values obtained during decoding. According to one possibility, if the performance decreases, the control module 7 can increase the quality of the extracted features to improve the precision. As shown in FIG. 4, the electronic device may comprise a secondary measurement sensor 9a configured to acquire a control quantity GC, with the control module configured to determine the quality evaluation parameter by taking into account a performance indicator determined from a value of the control quantity. According to another possibility shown in dashed lines in the same figure, the electronic device may comprise a user interface 9b making it possible to collect control information IC from a user.

According to a first example, the secondary measurement sensor 9a can be a position sensor, or a plurality of position sensors configured to identify abnormal positions of elements controlled by the actuator.

According to a second example, the secondary measurement sensor 9a can be a movement sensor, or a plurality of movement sensors configured to analyze the movement of the effectors controlled by the actuator, such as the exoskeleton, or user limbs and discriminate deviations from known scenarios such as walking, running, or grasping an object.

According to a third example, the user interface 9b can be a user interface sensor, such as a rheostat similar to a volume knob, which allows the user to give a value that corresponds to the quality evaluation parameter based on his perception.

In this case, the predefined range of quality values can correspond to a maximum value of the permissible deviation between the performance indicator and a nominal value, or to an area above a threshold value. For example, if a movement is controlled by the actuator and a measurement of this movement is carried out, the quality evaluation parameter could be a measured angle, and the range of quality values a predefined angular range to be achieved.

As an example, for feature extraction using the PLCWT method, considering that the precision control parameter corresponds to the number of approximation points, with values between 5 and 15 approximation points. The quality evaluation parameter can be defined as before as the deviation between the predefined angle and the measured angle, in degrees:

$$PEQ = \vert predefined angle - measured angle \vert$$

The determination of the precision control parameter is carried out, for example, in the following way:

$$If PEQ <= 10 , PCP = PCP - 1$$

$$If PEQ > 10 , PCP = min ( PCP + ( PEQ - 10 ) , 15 )$$

In this example, the range of quality values can be defined as PEQ<=10.

The PCP is proportional to the PEQ, but, depending on other applications, a more complex function with scaling or offsets may be required in order to establish the correspondence between the PCP and the PEQ.

According to a fourth embodiment shown in FIG. 4, the control module 7 is configured to determine the quality evaluation parameter by taking into account an anomaly detection. For example, this anomaly detection can be based on the comparison between the values or amplitudes of the extracted features S and a reference RS of expected values that can be extracted from a reference base 10 comprised in the electronic device 1 or accessible through this device. Each reference RS can be determined, for example, during the learning, so as to match an output state of the primary decoder 4 to expected values for the extracted features. For example, the reference RSs may correspond to ranges of values. Such a range of values RS can be deduced by considering the distribution of the values of the extracted feature S during training, for example by calibrating a Gaussian distribution based on the observed values, and by defining a range of values as being centered on the mean and having a width corresponding to a multiple or submultiple of the standard deviation of the distribution.

Thus, depending on the value of the command C or the state determined by the primary decoder, the control module can access the corresponding references RS in the reference base 10 which allow to know the expected values of the features.

FIG. 12 illustrates an example embodiment of the anomaly detection. The values of the features S, specifically features S0 to S4, are represented by black dots. In practice, the values of these extracted features S can correspond to spectral energy or power values in different frequency bands.

Thus, in the example in FIG. 12, the control module can compare the value of each extracted feature S with the corresponding reference RS, which might be, for example an expected range of values relative to the considered state or command C. In this example, the value for S1 (which might correspond to a given frequency, for example, 10 Hz) falls outside the reference or expected range RS1 for the considered state or control value. Under these conditions, the control module can be configured to determine the quality evaluation parameter PEQ, for example, as a vector comprising indications on potentially detected anomalies on each component or on the degree of correspondence with the reference RS, and to modify the precision control parameter PCP accordingly. Specifically, in the example in FIG. 12, the control module could be configured to modify the precision control parameter specifically for the feature S1 in order to increase the computation precision level of that feature. If there are no anomalies or if the observed values have a high degree of conformity with the reference, it is possible to decrease the precision control parameter to test a less energy-intensive computation.

Anomaly detection can be carried out by frequency band or by extracted feature. Thus, the control module is configured to identify the frequencies or the features involved in a given task that require improvement. According to one possibility, the control module can improve the quality of the extracted features for the specific identified frequencies.

In this embodiment, the predefined range of quality values can correspond to a maximum value of the permissible deviation between the values of the quality evaluation parameter and the references RS.

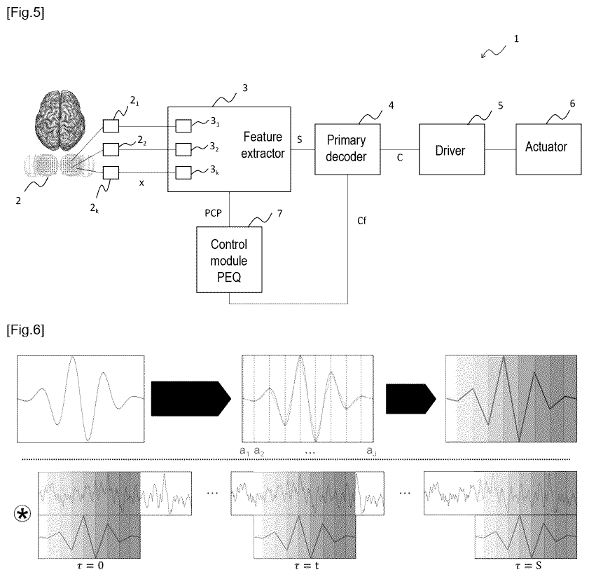

According to a fifth embodiment shown in FIG. 5, the control module is configured to determine the quality evaluation parameter by taking into account a confidence level Cf or probability value of a result of the primary decoder. These arrangements allow the probabilities provided by the primary decoder to be used as the confidence level for the issued decision. For example, decoders such as linear classifiers or decision trees associate a probability with a selected majority class. This probability could be used to control the quality level of the extracted features. According to one possibility, the control module can be configured to associate the confidence level or probability of a decoded class with a quality level required for the next decoding, and thus determine the precision control parameter PCP at each step. According to another possibility, the control module can be configured to determine a quality of the decision of the decoder and incrementally adjust the precision control parameter. The quality of the decision can be estimated, for example, by thresholding the probability of the decision. If the decision has a high level of confidence—it can therefore be considered over-satisfactory—the control module can be configured to incrementally decrease the feature extraction precision control parameter. Conversely, if the decision has a low level of confidence—it can therefore be considered questionable—the control module can be configured to incrementally increase the feature extraction precision control parameter.

In this embodiment, the predefined range of quality values can correspond to a maximum value of the permissible deviation between the values of the quality evaluation parameter and the references RS for a given state or control value.

According to some embodiments described above, it is possible that the quality evaluation parameter PEQ corresponds to a single value for all extracted features S and/or for all precision control parameters PCP.

This value can be binary, taking only two values (positive or negative feedback). In this case, the predefined range of quality values corresponds to only the positive value.

This value can also be a discrete value or a continuous value. In this case, the range of quality values can, for example, be defined as an area above a threshold.

Thus, according to one possibility, the control module can be configured to determine the precision control parameter PCP for the extraction by taking into account a set of ordered possible configurations of the extraction precision control parameter PCP. For example, in the case of an extraction of features of the approximate wavelet transform type PLCWT across multiple frequency bands, it is possible to define values of the precision control parameter PCP for the different frequencies according to several configurations. For example, it is possible to predict the values of subsequent subdivisions or numbers of points for several frequency values. To illustrate this principle, two frequency values at 10 Hz and 180 Hz can be isolated, the possible configurations being:
Configuration 1: 10 Hz=5 points; 180 Hz=25 points
Configuration 2: 10 Hz=10 points; 180 Hz=35 points
Configuration 3: 10 Hz=15 points; 180 Hz=45 points
Configuration N: computation of the exact CWT

By taking into account the current value of the extraction precision control parameter PCP which corresponds to one configuration among the set of possible configurations of the extraction precision control parameter, it is possible to change the configuration by incrementing to switch from one configuration to the next in one direction or the other based on the value of the extraction quality control parameter PEQ which thus defines the transition from the current value of the extraction precision control parameter to a new value of the extraction precision control parameter.

Thus, by identifying, according to the first embodiment, a dissatisfaction with the use as feedback or quality control parameter PEQ, if the extraction precision control parameter PCP corresponded to the configuration 2, the control module can modify the quality parameters to switch to the configuration 3 to increase the quality of the extraction of the features S.

According to another possibility, the control module 8 can be configured to modify the precision control parameter PCP of the extraction based on feedback concerning the quality control parameter PEQ by taking into account a current value of the extraction precision control parameter PCP corresponding, for example, to a given configuration of subdivision parameters/number of points per frequency, and a modification of the extraction precision control parameter, for a subset of features extracted which is random (with round-robin priority management) or predefined or based on learned or predefined heuristics depending on the extraction quality control parameter to increase or decrease the quality of the extraction.

Thus, according to the two possibilities mentioned above, the quality control parameter PEQ would allow the selection of a configuration and therefore the computation power necessary for a good compromise between cost and decoding precision.

The electronic device 1 may comprise one or more integrated circuits which comprise the feature extractor 2, the control module 3, the primary decoder 4, and optionally the secondary decoder 8.

The various embodiments of the electronic device described above allow the implementation of a method for extracting features from a measurement signal x as shown in FIG. 13, comprising:
A step of acquiring Et1 a measurement signal;
A step of extracting Et2 features from the measurement signal;
A decoding step Et3 using the extracted features S in order to determine a control signal C of an actuator;
In which the step of extracting features from the measurement signal is carried out approximately by taking into account a precision control parameter PCP, the method further comprising:
A step of determining Et4 (by computation, measurement or collection) a quality evaluation parameter PEQ;
A step of adjusting Et5 the precision control parameter (PCP) based on a value of the quality evaluation parameter.

The device and the method described above can be applied, as previously mentioned, to Brain-Computer Interfaces (BCIs) in order to improve the energy efficiency of the systems BCI, enabling less energy-intensive and portable applications.

The device and the method described above can also be applied to biomedical signal processing: The ability to dynamically adjust the quality of the extracted features can be applied to other fields of biomedical signal processing, such as EMG (electromyography) or electrocardiography (ECG).

The device and the method described above can be applied to seizure control systems in order to analyze brain signals in real time and detect seizures with increased precision while minimizing energy consumption.

The device and the method described above can be applied to other fields where extraction of features from signals is carried out.

1. An electronic device comprising:
a primary sensor configured to carry out the acquisition of a measurement signal of at least one physiological parameter of a living being;
a feature extractor configured to extract a feature from the measurement signal;
a primary decoder using the extracted feature and configured to determine a control signal;
an actuator with which the living being interacts;
a drive module for the actuator configured to drive the actuator based on the control signal determined by the primary decoder;
wherein the feature extractor is configured to be able to carry out the extraction of features from the measurement signal in an approximate manner according to several levels of precision, the required level of precision being a function of a desired precision control parameter, the electricity consumption of the feature extractor increasing with the level of precision;
the device further comprising a control module configured to determine a quality evaluation parameter of the action of the actuator in interaction with said living being;
the control module being configured to adjust the precision control parameter based on a value of the quality evaluation parameter by choosing said precision control parameter corresponding to the lowest level of precision allowing the value of the quality evaluation parameter to tend towards a predefined range of quality values or to maintain the value of the quality evaluation parameter within a predefined range of quality values.

2. The electronic device according to claim 1, wherein the range of quality values is defined by a determined deviation between the current quality evaluation parameter and the quality evaluation parameter that would be obtained with a full-precision computation.

3. The electronic device according to claim 1, wherein the measurement signal is an electrophysiological signal, in particular an electrophysiological signal emitted by the cortex.

4. The electronic device according to claim 1, comprising an integrated circuit which comprises at least one among the feature extractor, the control module, and the primary decoder.

5. The electronic device according to any of the preceding claim 1, wherein the actuator is an element among a spinal cord stimulator, a muscle stimulator, an exoskeleton actuator, a speech generator, a wheelchair actuator, such as a wheelchair motor, a display.

6. The electronic device according to claim 1, wherein the feature extractor is configured to carry out an approximate computation of a convolution between the input signal and a reference signal, taking into account the precision control parameter.

7. The electronic device according to claim 6, wherein the precision control parameter corresponds to a level of precision of an approximation function of the reference signal.

8. The electronic device according to claim 1, wherein the feature extractor is configured to extract a set of features from the measurement signal, the control module being configured to determine a single precision control parameter for several extracted features of the set of features, or to determine an individual precision control parameter for each of the features extracted from the set of features.

9. The electronic device according to claim 8, wherein the control module is configured to determine a single quality evaluation parameter for several extracted features of the set of features, or to determine an individual quality evaluation parameter for each of the extracted features of the set of features.

10. The electronic device according to claim 9, wherein the plurality of extracted features corresponds to a plurality of frequencies and/or a plurality of spatial locations.

11. The electronic device according to claim 1, comprising a secondary decoder configured to provide a user satisfaction signal, the quality evaluation parameter taking into account the user satisfaction signal.

12. The electronic device according to claim 1, wherein the control module is configured to periodically carry out or control an exact feature extraction computation and to measure a deviation between the result obtained with an exact feature extraction and that obtained with an approximate feature extraction with the level of the current extraction precision control parameter so as to determine a value of the quality evaluation parameter.

13. The electronic device according to claim 1, wherein the control module is configured to determine the quality evaluation parameter by taking into account a value of a performance indicator, the device comprising a secondary measurement sensor configured to acquire a control quantity or a user interface configured to collect control information from a user, the control module being configured to determine the quality evaluation parameter by taking into account the performance indicator determined from a value of the control quantity or the control information.

14. The electronic device according to claim 1, wherein the control module is configured to determine the quality evaluation parameter by taking into account an anomaly detection taking into account the value of the control signal and the value of the extracted features.

15. The electronic device according to claim 1, wherein the control module is configured to determine the quality evaluation parameter by taking into account a value of a confidence level or probability of a result of the primary decoder.

16. The electronic device according to claim 1, wherein the control module is configured to determine the precision control parameter of the extraction, taking into account:
a set of ordered possible configurations of the extraction precision control parameter;
a current value of the extraction precision control parameter selected from the set of possible configurations of the precision control parameter;
an increment dependent on the extraction and/or decoding quality control parameter defining the transition from the current value of the precision control parameter to a new value of the precision control parameter among the set of ordered possible configurations of the precision control parameter.

17. The electronic device according to any of the preceding claim 1, wherein the control module is configured to determine the precision control parameter of the extraction, taking into account:
a current value of the extraction precision control parameter;
a modification of the extraction precision control parameter, for a subset of extracted features which is random or predefined or based on learned or predefined heuristics depending on the quality control parameter.

18. A method for extracting features from a measurement signal of at least one physiological parameter of a living being comprising:
a step of acquiring the measurement signal of the at least one physiological parameter of a living being;
a step of extracting features from the measurement signal;
a decoding step using the extracted features in order to determine a control signal of an actuator with which the living being interacts;
a step of driving the actuator based on the control signal wherein the step of extracting features from the measurement signal is carried out approximately by taking into account a precision control parameter, the method further comprising:
a step of determining a quality evaluation parameter;
a step of adjusting the precision control parameter based on a value of the quality evaluation parameter by choosing said precision control parameter corresponding to the lowest level of precision allowing the value of the quality evaluation parameter to tend towards a predefined range of quality values or to maintain the value of the quality evaluation parameter within a predefined range of quality values.
